(19) United States
(12) Patent Application Publication
(10) Pub. No.: US 2022/0401548 A1
(43) Pub. Date: Dec. 22, 2022

(54) SINDBIS VIRUS DNA-BASED VACCINE

(71) Applicant: National Yang Ming Chiao Tung University, Taipei (TW)

(72) Inventors: Chia-Lin HSU, Taipei (TW); Lih-Hwa Hwang, Taipei (TW); Jian Wen Heng, Taipei (TW)

(73) Assignee: National Yang Ming Chiao Tung University, Taipei (TW)

(21) Appl. No.: 17/843,425

(22) Filed: Jun. 17, 2022

Related U.S. Application Data
(60) Provisional application No. 63/211,582, filed on Jun. 17, 2021.

(51) Int. Cl. A61K 39/12; A61K 39/295; C12N 7/00; A61P 31/14; A61K 39/215

(52) U.S. Cl. CPC A61K 39/12; A61K 39/295; C12N 7/00; A61P 31/14; A61K 39/215; A61K 2039/70

(57) ABSTRACT

Provided is a vaccine composition including a recombinant DNA vaccine against a pathogen. The recombinant DNA vaccine includes an expression cassette operably linked to a promoter, and the expression cassette encodes a non-structural protein of a Sindbis virus and an antigenic protein of the pathogen. Also provided is a method of producing a protective immune response against a pathogen in a subject in need thereof by administering the vaccine composition to the subject.

### TECHNICAL FIELD

The present disclosure relates to a vaccine composition, and particularly to a composition comprising a Sindbis virus (SINV) DNA-based vaccine.

### SEQUENCE LISTING

The instant application contains a Sequence Listing which has been filed electronically in ASCII format and is hereby incorporated by reference in its entirety. Said ASCII copy, created on Jun. 17, 2022, is named 210852US-YC-Y21PC003 sequence listing.txt and is 38 KB in size.

### DESCRIPTION OF RELEVANT ART

Viral infection has been established and remains as a serious animal and human affliction. In case of coronaviruses (CoVs), severe acute respiratory syndrome (SARS) in 2003, Middle East respiratory syndrome (MERS) in 2012, and coronavirus disease 2019 (COVID-19) in 2019 have triggered a global pandemic in public health emergency. Accordingly, there is always an ongoing desire to develop a virus vaccine prepared for a potential epidemic or even pandemic.

Presently, there are four types of vaccines commonly used in prevention of viral infections: (1) subunit vaccines, such as those including at least a portion of pathogenic proteins with adjuvants; (2) live-attenuated virus vaccines; (3) virus vector vaccines; and (4) RNA vaccines. In general, vaccines can provide active acquired immunity against viruses by triggering antibody responses in vaccinators, and then preventing viruses from infecting host cells.

However, for completely protecting from viral infection, in addition to induction of antibody responses, the virus-infected cells shall also be further eliminated to ensure that the viruses are completely removed from the host, so as to prevent latent viral infection or even virus mutation.

Although the first type of vaccines as described above can effectively induce neutralizing antibodies against viruses, it still cannot effectively remove virus-infected cells from the host. For the second type of vaccines, it would require large-scale production of attenuated viruses with strict quality control. Further, adenoviruses, one common type of pathogens in the neighboring environment of human society, has been widely used as a vector for the third type of virus vaccines as described above and has been demonstrated to effectively trigger immune responses in a host; however, it is often accompanied inevitably by strong side effects due to its inherent nature. As for the fourth type of vaccines, the challenge is the stability. Unlike its stable cousin, DNA, the natural structure of mRNA is meant to be easily degraded once it has completed the delivery of required “messages” for cells to divide, fight diseases, or simply live. Accordingly, RNA vaccines would necessarily require stricter requirements for storage and transportation, which results in the inconvenience of the vaccination process and also hinders the spread of vaccines.

In view of the rapid spread and mutation of viruses, there exists an urgent need for a vaccine platform that can quickly produce stable vaccines which are capable of not only effectively triggering antibody responses but also eliminating infected host cells.

### SUMMARY OF THE INVENTION

Provided is a vaccine platform, which is applicable for producing different vaccines. Also, the vaccines produced by the platform is capable of effectively eliciting neutralizing antibodies and eliminating infected cells that exhibit excellent stability for vaccination.

In at least one embodiment, the present disclosure provides a Sindbis virus DNA-based vaccine, which effectively produces antigens in the host through RNA replicons and includes complete protection against viral infections. Sindbis virus (SV) is a single-stranded RNA virus that causes asymptomatic infection and can be cleared out within a week in most of the infected subjects. Because of the instability of single-stranded RNAs, the SV genome is first reverse-transcribed and then cloned into a plasmid. The resulting plasmid, named “dSinC,” contains the non-structural polypeptides for SV replication and enables the transfected cells to produce a large quantity of RNA replicons expressing target antigens. Due to the lack of SV structural proteins, dSinC-transfected cells would not produce SV virions.

In the methods of the present disclosure, the immunizing components can be administered once or more than once (i.e., multiple times) to a subject. For example, a first immunizing component and/or a second immunizing component of the present disclosure can be administered one, two, three, four, five, six, seven, eight, nine, or ten times at any time interval (e.g., hours, days, weeks, months, years, etc.) and in any of the amounts described herein, which can be the same amount each time or different amounts at different times of administration in any combination. In other embodiments, the administration of the first and second immunizing components can be combined or arranged in any order (e.g., the first and second immunizing components can be administered in an alternating sequence or in any other order).

In at least one embodiment, the present disclosure provides a Sindbis virus DNA-based vaccine which may or may not include an adjuvant. Upon preparation of a Sindbis virus DNA-based vaccine composition, the adjuvant can be an aluminum, or an oil capable of forming an emulsion.

According to the RNA replicons from the Sindbis virus, the present disclosure may enforce the production of target antigens (e.g., the Spike protein of SARS-CoV-2) in the host cells. In addition, the RNA replicons per se can be used as a “danger signal” for innate immunity, and the target antigen can be further presented via major histocompatibility complex (MHC) class I. Accordingly, the present disclosure enables the recipients of the DNA-based vaccine to develop antibody response as well as T cell response to remove the infected cells.

### BRIEF DESCRIPTION OF THE DRAWINGS

The present disclosure can be more understood by reading the following descriptions of the embodiments, with reference made to one or more of the accompanying drawings below.

FIG. 1 is a schematic showing the dSinC-Spike plasmid.

FIG. 2 is a schematic showing the immunization strategy to test the a Sindbis virus DNA-based vaccine efficacy in mice.

FIG. 3 is a bar graph illustrating the titer of anti-Spike IgG in young adult mice measured by ELISA, upon immunization.

FIG. 4 is a line graph illustrating the production of neutralizing anti-Spike antibodies in young adult mice upon immunization.

FIG. 5 is a line graph illustrating the elicitation of anti-Spike IgG in aged mice upon 5 μg dose of Sindbis virus DNA-based vaccine. The 24 weeks old (aged) mice were immunized with the vaccine and tested for the production of anti-Spike IgG throughout a time course of pre-immunization to 8-week post-immunization.

FIG. 6 is a line graph illustrating viral neutralization response in young adult and aged mice upon 5 μg dose of Sindbis virus DNA-based vaccine. The 6-8 weeks old (young adult) and 24 weeks old (aged) mice were immunized with the vaccine and tested for the generation of neutralizing antibody response throughout a time course of pre-immunization to 8-week post-immunization.

FIGS. 7A-7B are graphs illustrating elicitation of anti-Spike IgG in obese adult mice upon immunization. FIG. 7A is a line graph illustrating body weight (g) of control vs. obesity group to validate the obesity model. FIG. 7B is a line graph illustrating measurement of anti-Spike IgG in obesity model upon immunization with 25 μg dose of Sindbis virus DNA-based vaccine.

FIG. 8 is a line graph illustrating viral neutralization response in obesity model upon immunization with 25 μg dose of vaccine. The 18 weeks old obese mice were immunized with the vaccine and tested for the generation of neutralizing antibody response throughout a time course of pre-immunization to 8-week post-immunization.

### DETAILED DESCRIPTION OF THE INVENTION

In the following descriptions of the embodiments of the present disclosure, reference is made to the accompanying drawings, which are shown to illustrate the embodiments in which the present disclosure may be practiced. These embodiments are provided to enable those skilled in the art to practice the present disclosure. It is understood that other embodiments may be used and that changes can be made to the embodiments without departing from the scope of the present disclosure. The following descriptions are therefore not to be considered as limiting the scope of the present disclosure.

It should also be understood that, unless clearly indicated to the contrary, in any methods claimed herein that include more than one step or act, the order of the steps or acts of the method is not necessarily limited to the order in which the steps or acts of the method are recited.

Titles or subtitles may be used in this disclosure for the convenience of a reader, which shall have no influence on the scope of the present disclosure.

Unless otherwise defined herein, scientific and technical terminologies employed in the present disclosure have the meanings that are commonly understood and used by one of ordinary skill in the art. All definitions, as defined and used herein, should be understood to control over dictionary definitions, definitions in documents incorporated by reference, and/or ordinary meanings of the defined terms.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The practice of the present disclosure employs, unless otherwise indicated, conventional techniques of molecular biology (including recombinant techniques), microbiology, cell biology, biochemistry, immunohistochemistry and immunology, which are well within the purview of a skilled artisan in the art. Such techniques are explained fully in the literature, such as “Molecular Cloning: A Laboratory Manual,” second edition (Sambrook, et al., 1989), Cold Spring Harbor Press; “Oligonucleotide Synthesis” (M. J. Gait, 1984); “Methods in Molecular Biology,” Humana Press; “Cell Biology: A Laboratory Notebook” (J. E. Cellis, ed., 1998) Academic Press; “Animal Cell Culture” (R. I. Freshney, ed., 1987); “Handbook of Experimental Immunology” (Weir, 1996); “Introduction to Cell and Tissue Culture” (J. P. Mather and P. E. Roberts, 1998); “Cell and Tissue Culture: Laboratory Procedures” (A. Doyle, J. B. Griffiths, and D. G. Newell, eds., 1993-8); “Methods in Enzymology” (Academic Press, Inc.); “Handbook of Experimental Immunology” (D. M. Weir and C. C. Blackwell, eds.); “Gene Transfer Vectors for Mammalian Cells” (J. M. Miller and M. P. Calos, eds., 1987); “Current Protocols in Molecular Biology” (F. M. Ausubel, et al., eds., 1987); “PCR: The Polymerase Chain Reaction (Mullis, et al., eds., 1994); “Current Protocols in Immunology” (J. E. Coligan et al., eds., 1991); “Short Protocols in Molecular Biology” (Wiley and Sons, 1999); “Immunobiology” (C. A. Janeway and P. Travers, 1997); “Antibodies” (P. Finch, 1997); “Antibodies: a practical approach” (D. Catty., ed., IRL Press, 1988-1989); “Monoclonal antibodies: a practical approach” (P. Shepherd and C. Dean, eds., Oxford University Press, 2000); “Using antibodies: a laboratory manual” (E. Harlow and D. Lane, Cold Spring Harbor Laboratory Press, 1999); The Antibodies (M. Zanetti and J. D. Capra, eds., Harwood Academic Publishers, 1995).

In some embodiments of the present invention, the first and/or second immunizing component can be administered with an adjuvant. As used herein, the term “adjuvant” refers to any substance that may enhance, improve, or otherwise modulate an immunological response in a host in addition to an antigen protein without deleterious effect on the subject. An adjuvant of the present invention can be, but is not limited to, for example, an immunostimulatory cytokine (including, but not limited to, interleukin-2, interleukin-12, interferon-gamma, interleukin-4, tumor necrosis factor-alpha, interleukin-1). Suitable adjuvants also include oil-in-water, saponin, an aluminum salt such as aluminum hydroxide gel (alum), aluminum phosphate, or algannmulin, but may also be a salt of calcium, iron or zinc, or may be cationically or anionically derivatized polysaccharides.

An adjuvant of the present disclosure can be administered before, concurrent with, and/or within a few hours, several hours, and/or 1, 2, 3, 4, 5, 6, 7, 8, 9, and/or 10 days before or after the administration of a composition of this invention to a subject. The effectiveness of an adjuvant or combination of adjuvants can be determined by measuring the immune response directed produced in response to administration of a composition of this invention to a subject with and without the adjuvant or combination of adjuvants, using standard procedures, as described herein and as known in the art. The term “pharmaceutically acceptable carrier” as used herein refers to any and all solvents, dispersion media, antibacterial and antifungal agents, isotonic and absorption delaying agents and the like, which may be appropriate for administration of the vaccine composition of the present disclosure. The pharmaceutically acceptable carrier useful for the present disclosure may include, but not be limited to, a preservative, a suspending agent, a tackifier, an isotonicity agent, a buffering agent, a humectant, and any combination thereof.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. If needed, various antibacterial and antifungal agents can be used, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA.

Dosage—An effective amount of the therapeutic or preventive agent is determined based on the intended goal, for example stimulation of an immune response against a viral infection. Those of skill in the art are well aware of how to apply gene delivery in vivo and ex vivo situations. For viral vectors, one generally will prepare a viral vector stock. Depending on the kind of virus and the titer attainable, one will deliver at least about, at most about, or about 1×10⁴, 1×10⁵, 1×10⁶, 1×10⁷, 1×10⁸, 1×10⁹, 1×10¹⁰, 1×10¹¹ or 1×10¹² infectious particles, or any value or range there between, to a subject. In some embodiments, Sindbis virus DNA-based vaccine according to the present disclosure may be administered in a single administration or multiple administrations.

The quantity to be administered, both according to number of treatments and dose, depends on the subject to be treated, the state of the subject and the protection desired. Precise amounts of the therapeutic composition also depend on the judgment of the practitioner and are peculiar to each individual.

As used herein, the term “vaccine” refers to a material capable of producing an immune response. A vaccine is typically understood to be a prophylactic or therapeutic material providing at least one antigen or antigenic function. The antigen or antigenic function may stimulate the body's adaptive immune system to provide an adaptive immune response.

As used herein, the term “expression” refers to the realization of genetic information encoded in a gene to produce a gene product such as an unspliced RNA, an mRNA, a spliced variant mRNA, a polypeptide, a protein, a post-translationally modified polypeptide, a spliced variant polypeptide and so on.

As used herein, the terms “administering,” “administered” or “administration” are used interchangeably to refer to a mode of delivery, including, without limitation, orally, topically, mucosally, transdermally and parenterally or through nebulization providing the active ingredient of the present disclosure to a subject in vivo, or in vitro or ex vivo to cells, tissues, or organs. Systemic administration is a route of administration of an agent into the circulatory system so that the entire body is affected. Administration can take place via enteral administration (absorption through the gastrointestinal tract) or parenteral administration (injection, infusion, or implantation).

Unless otherwise indicated, the term “subject,” “individual” or “patient” may be used interchangeably in the present disclosure, and refers to any animal. The animal can be a human subject or a non-human subject. The subject may be a human, but may also be a mammal, e.g., domestic animals, game animals, farm animals, and laboratory animals (e.g., rats, mice, guinea pigs, primates, and the like). The animal may be a non-human mammal, such as a non-human primate. Non-human primates include, but are limited to, chimpanzees, cynomolgus monkeys, spider monkeys, and macaques, e.g., Rhesus or Pan. Domestic animals and game animals include cows, horses, pigs, sheep, deer, bison, buffalo, mink, felines (e.g., domestic cats), canines (e.g., dogs), wolf, fox, avian species (e.g., chicken, turkey, and ostrich), and fish (e.g., trout, catfish, and salmon).

The following examples utilized the dSinC plasmid to develop a vaccine against COVID-19. Without further elaboration, it is believed that one skilled in the art can utilize the present disclosure to its fullest extent. The following embodiments are, therefore, to be construed as merely illustrative, and not limitative of the remainder of the disclosure in any way whatsoever.

dSinC-Spike Construction and Preparation

Codon optimized full SARS-CoV-2 spike sequence was amplified from pCMV-Spike (Sino Biological: VG40589-UT) (SEQ ID NO. 1) and fused with 3×FLAG-tag sequence (SEQ ID NO. 2) at C-terminus by PCR. The PCR fragment was inserted into a dSinC vector containing Sindbis virus genome (SEQ ID NO. 3) through the cloning sites MluI and StuI. The sequence of the resulting dSinC-Spike plasmid was confirmed by Sanger sequencing at National Yang Ming Chiao Tung University (NYCU) genome research center. The sequence of dSinC-Spike insert, which comprises the sequences of nonstructural protein, Psg, and capsid of Sindbis virus, SARS-CoV-2 spike, and 3×FLAG-tag, in the dSinC-Spike plasmid was represented by SEQ ID NO. 4.

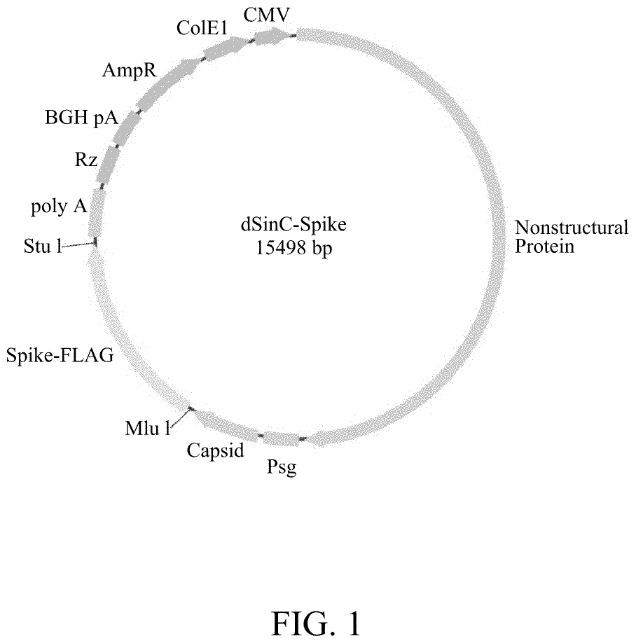

The confirmed dSinC-Spike plasmid was then transformed into competent E. Coli DH5a for expansion, and prepared using Midi prep (Geneaid Cat #PIE25) following the manufacturers' instructions. The dSinC-Spike plasmid was illustrated in FIG. 1, wherein the four SV non-structural proteins (nsP1 to nsP4) and their cleavage intermediates were involved in RNA replication. The dSinC-Spike plasmid was then sequenced and expanded for the following in vivo experiments.

Mouse Immunization

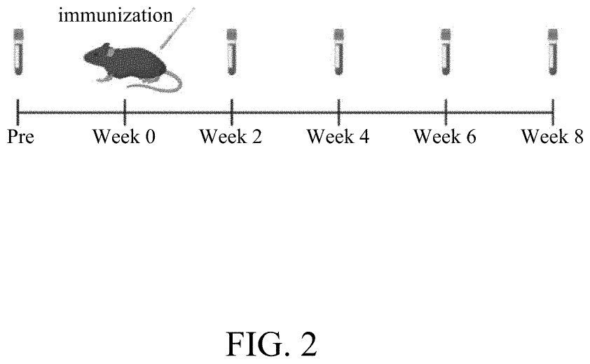

Six to eight weeks old male C57BL/6 housing in NYCU animal specific-pathogen-free (SPF) facility were used in this study. The dSinC-Spike plasmid was delivered intramuscularly (i.m.) with electroporation once throughout the study. For example, the mice were first anaesthetized by 4% isoflurane, and the hind leg was shaved and then injected with 30 μL of dSinC-Spike plasmid DNA in phosphate buffered saline (PBS) into the tibialis anterior muscles. After injection, the in vivo electroporation was applied using a BTX ECM 830 Pulse Generator. The serum samples were harvested every two weeks until 8 weeks post immunization. The protocol for dSinC-Spike immunization and blood sampling was illustrated in FIG. 2.

Spike-Specific IgG Enzyme-Linked Immunosorbent Assay (ELISA)

Blood samples were collected from mice every 2 weeks after immunization until week 8, the pre-bleed (Pre) before immunization served as negative control. The serum was harvested by allowing the whole blood samples to set at room temperature (RT) for 30 min, and then centrifuged at 2,000×g at 4° C. for 10 min. The resulting serum samples were stored at −80° C. until analysis.

An ELISA assembled in the lab was used to detect the spike-specific IgG titer of each time point. In brief, an ELISA plate was coated with recombinant Spike protein (1 μg/mL) (SinoBiological, Cat #40589-V08B1) at 4° C. overnight and blocked with PBS containing 1% bovine serum albumin (BSA) at room temperature (RT) for 1 hour before the assay. Samples were added to the well and incubated at RT for 2 hours. The serial diluted commercial Spike-specific antibody (SinoBiological Cat #40592-MM57) was used to generate the standard curve. At the end of incubation, the plates were washed with PBS containing 0.05% Tween-20 for three times. For detection, 0.16 μg/mL anti-mouse IgG with horseradish peroxidase (HRP) (JacksonImmuno Code: 115-035-166) was added and incubated at RT for 2 hours. At the end of reaction, 3,3′,5,5′-tetramethylbenzidine (TMB) substrate (Biolegend Cat #421101) was added for colorimetric development, with 2 N H₂SO₄ to stop the reaction. The samples were subjected to ELISA reader to measure absorbance at 450 nm.

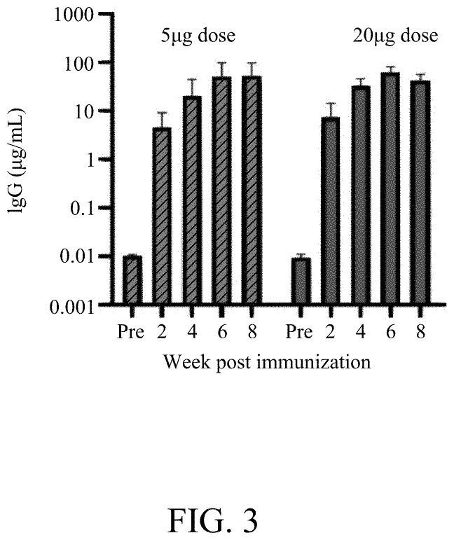

The results were shown in FIG. 3, indicating that 5 μg of DNA was sufficient to elicit as strong response as 20 μg DNA group, and about 10⁴-fold increase in anti-Spike IgG concentration in the mice received the immunization started at week 4 post immunization. The high anti-Spike IgG could be continuously detected until week 8 post immunization, suggesting a long-lasting response and protection for the recipients.

Pseudovirus Neutralization Assay

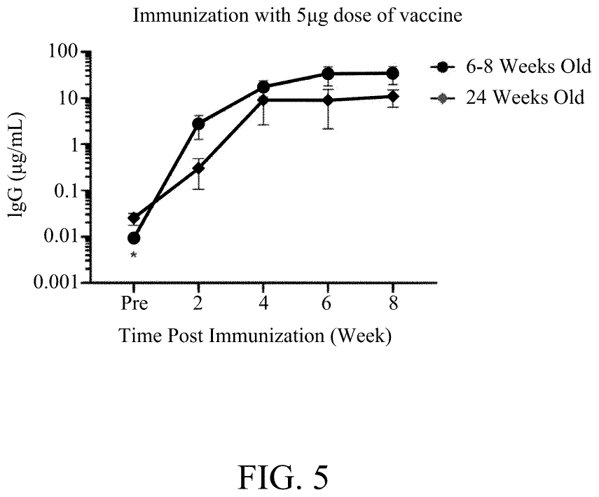

The SARS-CoV-2 spike pseudovirus and hACE2-293T cell line were obtained from RNA Technology Platform and Gene Manipulation Core, Taipei, Taiwan. The SARS-CoV-2 spike pseudovirus was produced by 293T cells and the titer of pseudovirus (TU) was determined based on cell viability of transduced A549 cells after puromycin selection. 1000 TU/μL means 1 μL of virus suspension could infect 1000 cells. hACE2-293T is a 293T cell line that expresses human ACE2 generated by lentivirus transduction and blasticidin selection, lentivirus plasmid was shown in appendix FIG. 5. hACE2-293T cells (1.25×104 per well) were seeded in 96-well white plate (Greiner bio-one #655083) and incubated at 37° C. with 5% CO2 overnight. Next day, 2.5-fold serial diluted serum sample and pseudovirus suspension in 150 μL cDMEM (final concentration 10 TU/μL) were mixed in a round bottom 96-well plate (Thermo #163320) and incubated at 37° C. for 1 hour. After incubation, 100 μL of serum and pseudovirus mixture were transferred to 96-well white plate to infect hACE2-293T cells, incubated at 37° C. for 48 hours. Finally, 100 μL of Bright-Glo™ Luciferase Assay reagent (Promega #E2620) was added into each well to allow cell lysis by waiting at least 2 minutes, then the luciferase activity (RLU) was measured by TECAN Infinite 200 plate reader. The RLU was converted into inhibition % and each sample with its corresponding inhibition curve and IC₅₀ was calculated by GraphPad Prism, nonlinear regression, [Inhibitor] vs. normalized response.

While several embodiments have been described and illustrated herein, those of ordinary skill in the art will readily envision a variety of other means for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the applications for which this disclosure is used. Those skilled in the art will recognize, or be able to ascertain using no more than routine experimentation, many equivalents to the embodiments described herein. It is, therefore, to be understood that the foregoing embodiments are presented by way of example only and that, within the scope of the appended claims and equivalents thereto, embodiments may be practiced otherwise than as described and claimed.

### Example 1: Neutralization Response in Young Adult Mice Immunization with 5 μg or 25 μg of Vaccine

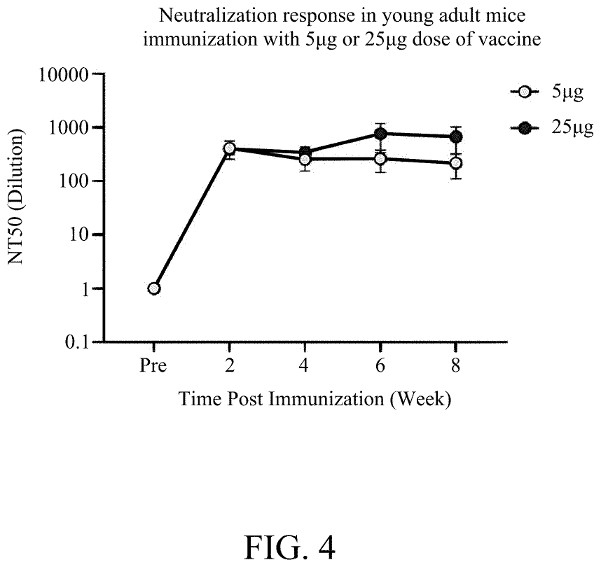

The 6 to 8 weeks old (young adult) mice were immunized with the 5 μg or 25 μg of vaccine and tested for the generation of neutralizing antibody response throughout a time course of pre-immunization to 8-week post-immunization. As shown in FIG. 4, there is an abundant NT50 response in young adult mice immunization with either 5 μg or 25 μg of vaccine.

### Example 2: Elicitation of Anti-Spike IgG in Aged Mice Upon Immunization

The 6 to 8 weeks old (young adult) and 24 weeks old (aged) mice were immunized with the vaccine and tested for the production of anti-Spike IgG throughout a time course of pre-immunization to 8-week post-immunization. As shown in FIG. 5, there is an abundant of IgG response in young adult and aged mice groups and the response sustained throughout at least 8 weeks after immunization with 5 μg of vaccine.

### Example 3: The Viral Neutralization Response in Vaccinated Aged Mice

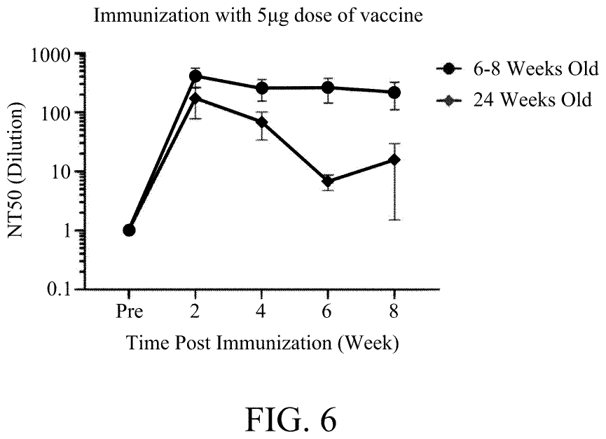

The 6 to 8 weeks old (young adult) and 24 weeks old (aged) mice were immunized with the vaccine and tested for the generation of neutralizing antibody response throughout a time course of pre-immunization to 8-week post-immunization. As shown in FIG. 6, there is a NT50 response in the young adult mice group and the aged mice group.

### Example 4: Elicitation of Anti-Spike IgG in Obese Adult Mice Upon Immunization

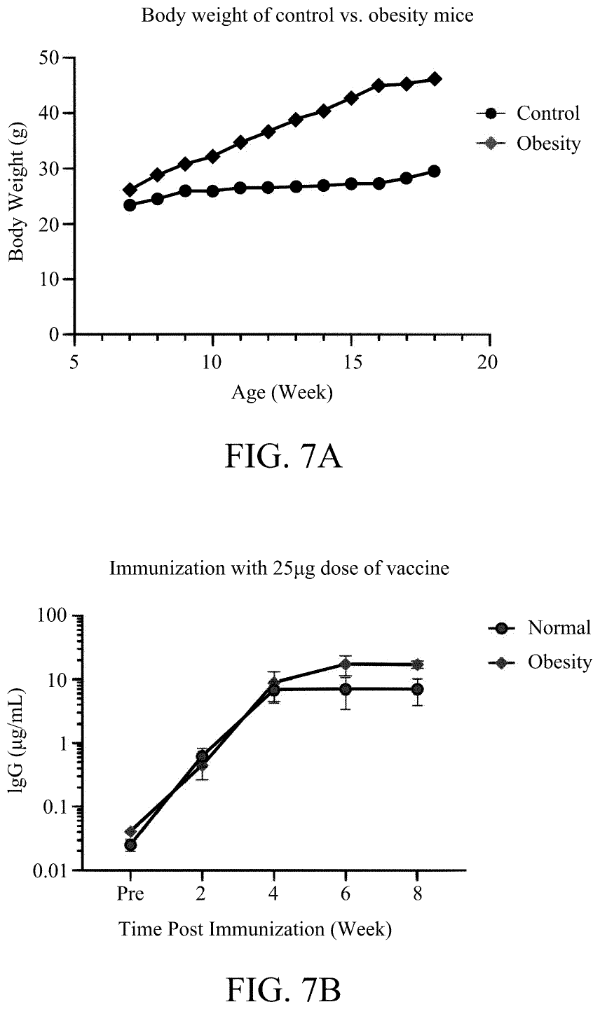

The young adult mice were fed with high-fat diet for 12 weeks to establish the obesity model. The obese mice were immunized with the vaccine and tested for the production of anti-Spike IgG throughout a time course of pre-immunization to 8-week post-immunization. As shown in FIG. 7B, there is an abundant of IgG response in both normal and obesity mice group and the IgG response sustained throughout at least 8 weeks after immunization with 25 μg of vaccine.

### Example 5: The Viral Neutralization Response in Vaccinated Obesity Mice

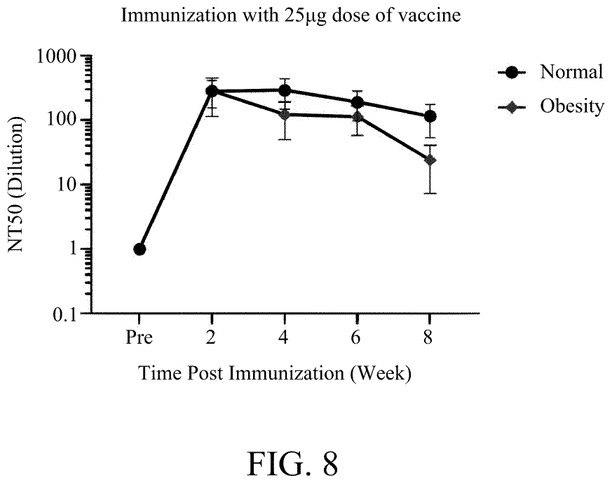

The 18 weeks old obese mice were immunized with the vaccine and tested for the generation of neutralizing antibody response throughout a time course of pre-immunization to 8-week post-immunization. As shown in FIG. 8, there is a NT50 response in the normal and obesity mice group.

What is claimed is:

1. A vaccine composition comprising a recombinant DNA vaccine against a pathogen and a pharmaceutically acceptable carrier, the recombinant DNA vaccine including an expression cassette operably linked to a promoter, wherein the expression cassette encodes a non-structural protein of a Sindbis virus and an antigenic protein of the pathogen.

2. The vaccine composition according to claim 1, wherein the expression cassette is free from expressing a structural protein of the Sindbis virus upon administration to a subject in need thereof.

3. The vaccine composition according to claim 2, wherein the antigenic protein of the pathogen produces a protective immune response against the pathogen upon expression in the subject.

4. The vaccine composition according to claim 1, wherein the non-structural protein of the Sindbis virus is at least one of nsP1, nsP2, nsP3, and nsP4.

5. The vaccine composition according to claim 1, wherein the pathogen is a bacterium or a virus.

6. The vaccine composition according to claim 5, wherein the virus is selected from the group consisting of influenza virus, severe acute respiratory syndrome coronavirus (SARS-CoV), Middle East respiratory syndrome coronavirus (MERS-CoV), SARS-CoV2, mouse hepatitis virus (MHV), porcine epidemic diarrhea virus (PEDV), respiratory syncytial virus (RSV), human immunodeficiency virus (HIV), hepatitis B virus (HBV), hepatitis C virus (HCV), Japanese encephalitis virus (JCV), Epstein Barr virus (EBV), and human papilloma virus (HPV).

7. The vaccine composition according to claim 1, wherein the promoter is a mammalian expression promoter.

8. The vaccine composition according to claim 1, wherein the promoter is selected from the group consisting of a cytomegalovirus (CMV) major immediate-early promoter, a simian virus 40 (SV40) promoter, a β-actin promoter, an albumin promoter, an elongation factor 1-α (EF1-α) promoter, a PγK promoter, an MFG promoter, and a Rous sarcoma virus promoter.

9. The vaccine composition according to claim 1, wherein the expression cassette is a linear or circular DNA molecule.

10. The vaccine composition according to claim 1, wherein the expression cassette further comprises a poly-A sequence.

11. The vaccine composition according to claim 1, wherein the expression cassette is codon optimized for expression in a eukaryotic cell.

12. The vaccine composition according to claim 11, wherein the eukaryotic cell is a yeast cell or a mammalian cell.

13. A method of producing a protective immune response against a pathogen in a subject in need thereof, comprising administering the vaccine composition of claim 1 to the subject.

14. The method of claim 13, wherein the recombinant DNA vaccine is administered to the subject 1 to 3 times within one year.

15. The method of claim 13, wherein the recombinant DNA vaccine is administered to the subject once every other month.

16. The method of claim 13, wherein the subject is a mammal.


SEQUENCE LISTING (4 sequences)

SEQ ID NO: 1 - DNA, 3822 bp, Artificial Sequence
  Synthetic sequence, sequence of codon optimized SARS-CoV-2 Spike (Sino Biological VG40589-UT
atgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctga

SEQ ID NO: 2 - DNA, 3906 bp, Artificial Sequence
  Synthetic sequence, sequence of inserted PCR product (SARS-CoV-2 Spike; 3xFLAG Tag)
atgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctctagaggatcccgggctgactacaaagaccatgacggtgattataaagatcatgacatcgactacaaggatgacgatgacaagtag

SEQ ID NO: 3 - DNA, 8388 bp, Artificial Sequence
  Sequence of Sindbis Virus Genome (Nonstructural protein + Psg + Capsid)
atggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaatagaggattctttaacatgctcggccgccgccccttcccggcccccactgccatgtggaggccgcggagaaggaggcaggcggccccgatgcctgcccgcaacgggctggcttctcaaatccagcaactgaccacagccgtcagtgccctagtcattggacaggcaactagacctcaacccccacgtccacgcccgccaccgcgccagaagaagcaggcgcccaagcaaccaccgaagccgaagaaaccaaaaacgcaggagaagaagaagaagcaacctgcaaaacccaaacccggaaagagacagcgcatggcacttaagttggaggccgacagattgttcgacgtcaagaacgaggacggagatgtcatcgggcacgcactggccatggaaggaaaggtaatgaaacctctgcacgtgaaaggaaccatcgaccaccctgtgctatcaaagctcaaatttaccaagtcgtcagcatacgacatggagttcgcacagttgccagtcaacatgagaagtgaggcattcacctacaccagtgaacaccccgaaggattctataactggcaccacggagcggtgcagtatagtggaggtagatttaccatccctcgcggagtaggaggcagaggagacagcggtcgtccgatcatggataactccggtcgggttgtcgcgatagtcctcggtggcgctgatgaaggaacacgaactgccctttcggtcgtcacctggaatagtaaagggaagacaattaagacgaccccggaagggacagaagagtggtccgcagca

SEQ ID NO: 4 - DNA, 12300 bp, Artificial Sequence
  Synthetic sequence, sequence of SINV-Spike insertion Sindbis Virus Genome (Nonstructural protein + Psg + Capsid); SARS-CoV-2 Spike; 3xFLAG Tag)
atggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaatagaggattctttaacatgctcggccgccgccccttcccggcccccactgccatgtggaggccgcggagaaggaggcaggcggccccgatgcctgcccgcaacgggctggcttctcaaatccagcaactgaccacagccgtcagtgccctagtcattggacaggcaactagacctcaacccccacgtccacgcccgccaccgcgccagaagaagcaggcgcccaagcaaccaccgaagccgaagaaaccaaaaacgcaggagaagaagaagaagcaacctgcaaaacccaaacccggaaagagacagcgcatggcacttaagttggaggccgacagattgttcgacgtcaagaacgaggacggagatgtcatcgggcacgcactggccatggaaggaaaggtaatgaaacctctgcacgtgaaaggaaccatcgaccaccctgtgctatcaaagctcaaatttaccaagtcgtcagcatacgacatggagttcgcacagttgccagtcaacatgagaagtgaggcattcacctacaccagtgaacaccccgaaggattctataactggcaccacggagcggtgcagtatagtggaggtagatttaccatccctcgcggagtaggaggcagaggagacagcggtcgtccgatcatggataactccggtcgggttgtcgcgatagtcctcggtggcgctgatgaaggaacacgaactgccctttcggtcgtcacctggaatagtaaagggaagacaattaagacgaccccggaagggacagaagagtggtccgcagcaacgcgtatgtttgtgttcctggtgctgctgccactggtgtccagccagtgtgtgaacctgaccaccaggacccaacttcctcctgcctacaccaactccttcaccaggggagtctactaccctgacaaggtgttcaggtcctctgtgctgcacagcacccaggacctgttcctgccattcttcagcaatgtgacctggttccatgccatccatgtgtctggcaccaatggcaccaagaggtttgacaaccctgtgctgccattcaatgatggagtctactttgccagcacagagaagagcaacatcatcaggggctggatttttggcaccaccctggacagcaagacccagtccctgctgattgtgaacaatgccaccaatgtggtgattaaggtgtgtgagttccagttctgtaatgacccattcctgggagtctactaccacaagaacaacaagtcctggatggagtctgagttcagggtctactcctctgccaacaactgtacctttgaatatgtgagccaaccattcctgatggacttggagggcaagcagggcaacttcaagaacctgagggagtttgtgttcaagaacattgatggctacttcaagatttacagcaaacacacaccaatcaacctggtgagggacctgccacagggcttctctgccttggaaccactggtggacctgccaattggcatcaacatcaccaggttccagaccctgctggctctgcacaggtcctacctgacacctggagactcctcctctggctggacagcaggagcagcagcctactatgtgggctacctccaaccaaggaccttcctgctgaaatacaatgagaatggcaccatcacagatgctgtggactgtgccctggacccactgtctgagaccaagtgtaccctgaaatccttcacagtggagaagggcatctaccagaccagcaacttcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttcaacttcaatggactgacaggcacaggagtgctgacagagagcaacaagaagttcctgccattccaacagtttggcagggacattgctgacaccacagatgctgtgagggacccacagaccttggagattctggacatcacaccatgttcctttggaggagtgtctgtgattacacctggcaccaacaccagcaaccaggtggctgtgctctaccaggatgtgaactgtactgaggtgcctgtggctatccatgctgaccaacttacaccaacctggagggtctacagcacaggcagcaatgtgttccagaccagggctggctgtctgattggagcagagcatgtgaacaactcctatgagtgtgacatcccaattggagcaggcatctgtgcctcctaccagacccagaccaacagcccaaggagggcaaggtctgtggcaagccagagcatcattgcctacacaatgagtctgggagcagagaactctgtggcttacagcaacaacagcattgccatcccaaccaacttcaccatctctgtgaccacagagattctgcctgtgagtatgaccaagacctctgtggactgtacaatgtatatctgtggagacagcacagagtgtagcaacctgctgctccaatatggctccttctgtacccaacttaacagggctctgacaggcattgctgtggaacaggacaagaacacccaggaggtgtttgcccaggtgaagcagatttacaagacacctccaatcaaggactttggaggcttcaacttcagccagattctgcctgacccaagcaagccaagcaagaggtccttcattgaggacctgctgttcaacaaggtgaccctggctgatgctggcttcatcaagcaatatggagactgtctgggagacattgctgccagggacctgatttgtgcccagaagttcaatggactgacagtgctgcctccactgctgacagatgagatgattgcccaatacacctctgccctgctggctggcaccatcacctctggctggacctttggagcaggagcagccctccaaatcccatttgctatgcagatggcttacaggttcaatggcattggagtgacccagaatgtgctctatgagaaccagaaactgattgccaaccagttcaactctgccattggcaagattcaggactccctgtccagcacagcctctgccctgggcaaactccaagatgtggtgaaccagaatgcccaggctctgaacaccctggtgaagcaactttccagcaactttggagccatctcctctgtgctgaatgacatcctgagcagactggacaaggtggaggctgaggtccagattgacagactgattacaggcagactccaatccctccaaacctatgtgacccaacaacttatcagggctgctgagattagggcatctgccaacctggctgccaccaagatgagtgagtgtgtgctgggacaaagcaagagggtggacttctgtggcaagggctaccacctgatgagttttccacagtctgcccctcatggagtggtgttcctgcatgtgacctatgtgcctgcccaggagaagaacttcaccacagcccctgccatctgccatgatggcaaggctcactttccaagggagggagtgtttgtgagcaatggcacccactggtttgtgacccagaggaacttctatgaaccacagattatcaccacagacaacacctttgtgtctggcaactgtgatgtggtgattggcattgtgaacaacacagtctatgacccactccaacctgaactggactccttcaaggaggaactggacaaatacttcaagaaccacaccagccctgatgtggacctgggagacatctctggcatcaatgcctctgtggtgaacatccagaaggagattgacagactgaatgaggtggctaagaacctgaatgagtccctgattgacctccaagaactgggcaaatatgaacaatacatcaagtggccatggtacatctggctgggcttcattgctggactgattgccattgtgatggtgaccataatgctgtgttgtatgacctcctgttgttcctgtctgaaaggctgttgttcctgtggctcctgttgtaagtttgatgaggatgactctgaacctgtgctgaaaggagtgaaactgcactacacctctagaggatcccgggctgactacaaagaccatgacggtgattataaagatcatgacatcgactacaaggatgacgatgacaagtag